(19) United States
(12) Patent Application Publication
(10) Pub. No.: US 2008/0039907 A1
(43) Pub. Date: Feb. 14, 2008

(54) Phototherapy Systems and Methods

(75) Inventor: Peter Depew Fiset, Loudonville, NY (US)

Correspondence Address: FOLEY AND LARDNER LLP;SUITE 500, 3000 K STREET NW, WASHINGTON, DC, 20007, US

(73) Assignee: LEDeep, LLC, Troy, NY (US)

(21) Appl. No.: 11/547,865

(22) PCT Filed: Apr. 12, 2005
(86) PCT No.: PCT/US05/12017

§ 371 Date: Oct. 6, 2006

Related U.S. Application Data
(60) Provisional application No. 60/561,797, filed on Apr. 12, 2004.

(51) Int. Cl. A61N 5/06; A61K 8/19; A61P 17/00; A61Q 17/04

(52) U.S. Cl. 607/94; 424/49; 607/88

(57) ABSTRACT

A phototherapy method includes applying to skin a lotion which includes a carrier material and nanocrystals located in the carrier material, and exposing the lotion to photons of a first wavelength range such that the nanocrystals convert the photons of a first wavelength range into photons of a second wavelength range suitable for phototherapy and provide the photons of the second wavelength range to the skin.

### CROSS-REFERENCE TO RELATED APPLICATIONS

The present application claims benefit of U.S. Provisional Patent Application Ser. No. 60/561,797, filed Apr. 12, 2004 incorporated by reference in its entirety.

### FIELD OF THE INVENTION

The present invention is directed generally to phototherapy methods and lotions containing nanocrystals suitable for phototherapy.

### BACKGROUND

Sunscreens use ultraviolet (UV) light blocking materials including but not limited to bulk zinc oxide (ZnO) powders in a carrier material for the purpose of blocking UV rays through the process of absorption and/or reflection. However, while these sunscreens block out harmful UV rays for some people, not all wavelengths of light are harmful. Thus, the use of UV protective lotions, such as sunscreens, has the benefit of reducing exposure to harmful UV rays with the drawback of reducing exposure to beneficial UV and other visible rays.

Light (i.e., radiation) with wavelengths in the ultra-violet range is often referred to as UV light or UV. UVA, UVB and UVC describe three separate non-overlapping but adjacent ranges of light fully encompassing the UV light range. The range of light referred to as UVA generally has the longest set of wavelengths within the UV range and includes wavelengths between 290 and 400. Generally, UVA-1 is between about 340 and about 400 nm; UVA-2 is between about 290 and about 340 nm, such as between about 310-315 to about 340 nm; and UVA-3 is between about 290 and about 310-315 nm. The range of light referred to as UVC generally has the shortest set of wavelengths within the UV range and includes wavelengths between 160 and 260. The range of light referred to as UVB includes wavelengths between 260 and 290.

The use of the terms UVA, UVB and UVC allow the various properties of UV light to be categorized in general ways. UVA has the best capability of tanning skin. UVB does not produce a tan in the third layer of skin. UVC light does not produce a tan but can sterilize some biological agents such as certain bacteria. Under certain conditions UVB will tan the second layer of skin. The second layer of skin when tanned with UVB has a shedding period of 5 to 8 days. Skin tanned with UVA only has the third layer of skin tanned which results in a normal shedding cycle of 28 days.

A light or photo therapy is a method of applying a specific set of wavelengths of electromagnetic radiation in specific states and under specific conditions to produce a change in a bodily function. Tanning is a light therapy whereby the biological change includes the production of melanin within the cells of the skin (i.e., the tanning cycle generally begins with UV and quiescent melanin production and continues with darkening process of the melanin through UV, such as UVA, irradiation). Indoor-tanning is a light therapy utilizing the exposure of moderate amounts of UV over a reasonable amount of time to skin from UV sources other than the sun.

Under normal conditions the outer layer of skin, also known as the first layer, is composed of dead cells. Normally, dead cells will not produce melanin upon exposure to moderate amounts of UV. The layer under the first layer of skin is referred to as the second layer of skin, and is composed of active cells that may be functioning in some biological manner and will produce melanin upon exposure to UVB light. UVB skin tanning has, what some tanners consider, an additional negative effect. UVB tanning will thicken the second layer of skin and as a result increases the visibility of skin lines and wrinkles. UVB tanning creates a shedding cycle of 5 to 7 days which is undesirable when a UVA tan has a shedding cycle of 28 days. When UVB is combined with UVA, the shedding cycle of the UVA tanned layer is accelerated since the second layer is shed more quickly, and the third layer becomes the second layer as a result, and is shed within another 5 to 7 days. Thus, some UV light wavelength ranges are more beneficial than others.

### BRIEF SUMMARY OF THE INVENTION

A phototherapy method includes applying to skin a lotion which includes a carrier material and nanocrystals located in the carrier material, and exposing the lotion to photons of a first wavelength range such that the nanocrystals convert the photons of a first wavelength range into photons of a second wavelength range suitable for phototherapy (including but not limited to tanning) and provide the photons of the second wavelength range to the skin.

### DETAILED DESCRIPTION OF THE PREFERRED EMBODIMENTS

The present inventor has realized that a topical lotion adapted to be applied to human skin can be used for phototherapy. The lotion includes a carrier material adapted to be applied to human skin and radiation emitting nanocrystals located in the carrier material. The nanocrystals are adapted to convert photons of a first wavelength range into photons of a second wavelength range suitable for phototherapy. In other words, the nanocrystals are adapted to absorb incident light which may include undesirable wavelength ranges and emit light (such as UV radiation or visible light) to the human skin in the desired wavelength range for phototherapy.

For some people, while some wavelength ranges of light are harmful, other wavelength ranges of light are beneficial. Different people may have differing reactions to various wavelengths of light. For example, many people benefit from exposure to UV light, which results in the production of Vitamin-D. Furthermore, as described above, UVA wavelength ranges are considered to be beneficial for tanning, while UVB and UVC wavelength ranges are generally considered not beneficial. In addition, certain people benefit from wavelengths of light outside the UV range. For example, orange light has been found to reduce wrinkles in aging skin.

The use of prior art UV protective lotions, such as sunscreens and sunblocks has the benefit of reducing exposure to harmfil UV rays with the drawback of reducing exposure to beneficial UV and visible rays. While the prior art lotions and creams contain materials which absorb and/or reflect UV light to block UV light from human skin, the lotion of the preferred embodiments of the invention changes or converts the wavelength(s) of the incident light for useful purposes.

An example of such a useful wavelength conversion includes the conversion of incident light containing wavelengths shorter than 340 nm to wavelengths longer than 340 nm for tanning and Lupus treatment. Another example of the wavelength conversion process is the conversion of incident light containing wavelengths longer and shorter than about 311 nm to wavelengths of about 311 nm for psoriasis phototherapy. It should be noted that a single phototherapy or a combination of phototherapies may be conducted using the above described lotion. For example, tanning and/or Lupus and/or other therapies may be conducted at the same time or sequentially on the same person using the above described lotion. Furthermore, the lotion may be used for multiple wavelength conversion of portions of the total photons within wavelength ranges for broadening spectral densities or other uses. For example, the lotion may contain two or more types of nanocrystals of different average sizes and/or composed of different materials. Each nanocrystal type emits light of a different peak wavelength. Thus, the combined nanocrystals may emit light having two or more different peak wavelengths. Furthermore, light emitted by one type of nanocrystals may be absorbed and reemitted as light having one or more different (such as longer) peak wavelength by the other type(s) of nanocrystals.

As used herein, phototherapy is a broad term which encompasses many and varied applications of light to the skin and internal organs. Phototherapy includes, but is not limited to, natural sun tanning, artificial (such as indoor) tanning, long wavelength UV (340 nm to 390 nm) treatment for Lupus, narrow UVB (308 nm to 313 nm) treatment for psoriasis and various other medical light treatments. It should be noted that for medical phototherapies, such as Lupus and psoriasis phototherapies, a therapeutically effective amount of lotion is provided or administered to the skin of the persons in need thereof (i.e., persons who suffer from these medical conditions or diseases) in order to treat these conditions or diseases. Furthermore, the lotion may be selectively provided to the portions of the skin that should be tanned and/or to portions of the skin which are affected by the medical condition or disease. For example, for Lupus therapy, preferably all or a major portion of a person's skin is covered by the lotion and exposed to incident light, since it has been previously indicated that irradiation of skin portion not covered by lesions is beneficial for decreasing lesions and improving internal organ function. In contrast, for psoriasis therapy, preferably only the affected skin is covered by the lotion and irradiated with incident light. If desired, the portions of the skin not covered by the lotion may be covered by clothing, sun block or other light blocking means to avoid exposing healthy and/or already tanned skin portions to incident light.

As used herein, the term “incident” light or radiation includes natural incident light, such as sunlight, and artificial light, such as lamp light, LED light, laser light, etc. The incident light or radiation includes broad spectrum radiation sources, such as sunlight, which include UVA, UVB, UVC and visible light components and narrow spectrum light, such as UV or visible LED or laser light having a narrow wavelength distribution.

Thus, the topical or dermatological lotion can be used for the conversion of one or more wavelengths or wavelength ranges of light including but not limited to UV, UVC, UVB, UVA, VIS, NIR, and FIR, light to a specific wavelength or wavelength range including but not limited to 311 nm, 308 nm to 313 run, and 340 nm-390 nm, for a useful purpose including but not limited to psoriasis phototherapy, Lupus therapy, skin tanning, and Vitamin-D enhancement phototherapy.

The lotion incorporating one or more selected nanocrystals can convert photons with wavelengths which may be unsuitable for phototherapy applications and/or harmful to humans into photons with wavelengths which are suitable for a phototherapy application or a combination of phototherapy applications. However, it should be noted that the lotion does not necessarily block or reflect all light (i.e., photons) having wavelengths which are unsuitable for phototherapy applications and/or harmful to humans and that the lotion does not necessarily convert this light to light which contains only wavelengths which are either suitable for phototherapy applications and/or not harmful to humans. In other words, the lotion may allow (to a limited therapeutically acceptable level) not useful and/or harmful wavelengths of light to reach the skin and the lotion may emit wavelengths of light which are not useful and/or harmful in addition to wavelengths which are useful for phototherapy. Preferably, the lotion emits light having a peak wavelength in the useful wavelength range and has a small portion, such as less than 10 to 20% of all emitted light when plotted on a graph of intensity versus wavelength, that is in the not useful and/or harmful range.

Any suitable light converting/emitting nanocrystals may be used in the lotion. It should be noted that the nanocrystals are also sometimes referred to as nanoparticles or quantum dots. For the purposes of the present invention, these terms should be considered to be interchangeable. Furthermore, nanocrystals or nanoparticles or quantum dots which are doped with activator ions which are responsible for optical emission are sometimes called nanophosphors.

Preferably, the nanocrystals comprise semiconductor nanocrystals. More preferably, the nanocrystals comprise semiconductor nanocrystals which emit UV light, such as UVA and/or 311 nm light upon absorbing (i.e., being irradiated with) incident light, such as sunlight and/or artificial UV and/or visible light. However, non-semiconductor nanocrystals, such as ceramic phosphor nanocrystals containing light emitting activator ions may also be used. Nanocrystals may have any suitable size, such as an average diameter less than 100 nm, such as a diameter of 2-20 nm, for example.

One non-limiting example of such nanocrystals comprise zinc oxide (ZnO) nanocrystals. Other nanocrystals, such as titanium dioxide nanocrystals, may be used instead of or in addition to ZnO nanocrystals. It should be noted that zinc oxide and titanium dioxide nanocrystals also include nanocrystals which are somewhat non-stoichiometric (i.e., do not have the exact 1:1 or 1:2 metal to oxygen ratio). The nanocrystals may comprise undoped nanocrystals which emit UV or other light due to their size, such as by absorbing incident light and emitting light due to exciton recombination. Alternatively, the nanocrystals may comprise doped phosphor nanocrystals which are doped with suitable activator ions which emit light having one peak wavelength when the nanocrystals absorb light containing shorter wavelengths. In contrast to bulk ZnO or TiO₂ powders or lotions containing bulk ZnO or titanium dioxide which block and/or reflect UV light, the nanocrystals emit UV light in response to absorbing incident light. The nanocrystals, such as ZnO nanocrystals, may be prepared by any suitable method and their average diameter and/or activator may be selected to provide light emission having a desired peak wavelength. For example, H. Zhou et al., Appl. Phys. Lett. 80 (2) (2002) 210, incorporated herein by reference in its entirety, describe a method of making ZnO light emitting nanocrystals, where the nanocrystals are subjected to a high temperature anneal at 300 to 500° C. to burn off a Zn(OH)₂ shell from the ZnO nanocrystals. The removal of the shell is reported to increase light emission from the ZnO nanocrystals.

The lotion also contains a carrier material or compound. Any suitable material in which the nanocrystals can be dispersed and which can be applied to human skin can be used. The carrier material or compound can include, but is not limited to, petrolatum, mineral oil, silicone oil, and petroleum jelly. Preferably, the carrier material has properties which include but are not limited to substantial transparency to UV light, nanocrystal suspension capabilities, and low toxicity. Alternatively, the carrier material may also comprise a material which evaporates after contact with skin, such as water, alcohols and/or fluorocarbon materials, such as hydrofluorocarbon and chlorofluorocarbon materials. Furthermore, it is possible that the carrier material may be omitted and the nanocrystals may be applied directly to skin in powder form.

Thus, one example of topical nanocrystal lotion is zinc oxide nanocrystals suspended in silicone oil carrier material. A further embodiment of the present invention is a topical nanocrystal lotion comprised of one or more nanocrystal compounds including but not limited to ZnO providing a useful purpose including but not limited to the conversion of photons of a first wavelength range into photons of a second wavelength range, and for use in providing arbitrary specific wavelength ranges for arbitrary phototherapy methods including but not limited to indoor tanning, scleroderma therapy, scleriasis therapy, lupus therapy, photopheresis, and photochemotherapy. Further, elemental molar concentrations of said nanocrystals are chosen to meet criteria set forth by phototherapy requirements, as will be described below. The properties of the nanocrystals that can be selected to meet phototherapy requirement include but are not limited to tuned dimensional size and tuned composition (including activator ion selection if activator ions are present).

While the lotion is preferably applied to human skin, it may also be applied to human hair to provide desired wavelength light to hair to improve hair growth and/or to provide desired wavelength light to skin under the hair. Furthermore, the lotion may also be applied to animal skin and/or hair to provide phototherapy for animals. Likewise, the nanocrystal composition may also be applied to inanimate objects if desired to covert light wavelengths being provided to inanimate objects, such as clothes, glass, plastic, photopheresis equipment, umbrellas, which convert the incident light to useful UV light (i.e., radiation) which is then provided or directed to the person or animal located adjacent to the object to provide one or more phototherapies for the person or animal. It should be noted that the inanimate objects should be positioned to either reflect the UV light onto the person or animal or they should be transparent to incident light and emitted UV light. For example, the umbrellas and clothes may be made of a UV transparent material or from a mesh type material. In case of photopheresis equipment, the nanocrystals are adapted to provide UV light to the patient's blood or to separated components of the patient's blood.

The lotion is preferably located in a container adapted to dispense the lotion onto human skin. The container may be part of a lotion delivery system comprised of one or more components including but not limited to a pressurized spray container with nozzle, a jar with lid, a tube with cap, a bag, such as a plastic bag, a flexible container with opening, a single use container, a mix upon use container, and a nanocrystal lotion impregnated tissue. The mix upon use container may have one or more chambers each filled with a component of the resulting discharged lotion that effectively mixes the nanocrystals with the carrier material as it exits the container. The pressurized spray container includes a hand-held spray container (such as a small container with a spray nozzle) as well as a container which is part of a larger pressurized lotion delivery system, such as a spray tanning chamber. An example of a spray tanning chamber includes automatic or manual spray tanning chambers which spray a person with DHA or other based tanning lotion. Such tanning chambers include open and closed chambers. In the present case, the spray tanning chamber would spray a lotion containing the nanocrystals, such as zinc oxide or titanium dioxide nanocrystals, onto the skin of a person located in the chamber. The carrier material of such a lotion may include DHA if desired. The spray chamber may contain a source of incident light for providing phototherapy in the chamber or it may lack a source of incident light and function as a lotion spray booth. The spray chamber may also optional include a hose, sprinkler or other means to clean the person with water or other fluid and nanocrystal waste recovery device which collects the nanocrystal waste with the run off.

A further embodiment of the present invention includes an applied topical lotion testing system useful for purposes including but not limited to determining the effective coverage of said topical nanocrystal lotion, tanning effectiveness, phototherapy effectiveness, historical trend of phototherapy application, feed forward control of phototherapy, feedback control of phototherapy, phototherapy session timing, erythemal metering, progress timing and/or safety consideration, such as consideration of various safety components. Said applied topical lotion testing system may incorporate a plurality of one or more components including but not limited to a spectrophotometer, diffraction grating, power supply, user interface, memory, storage, communication, computer interface, and/or safety equipment. In the instance wherein said safety components are interfaced with phototherapy incident light producing device, such as a lamp, to provide an automatic shutdown with set point limits including but not limited to maximum exposure set point and secondary condition set point. The secondary condition set point may include a second topical nanocrystal lotion, where said second topical nanocrystal lotion has the same or has a differing composition than said topical nanocrystal lotion depending upon the requirements of the phototherapy. If desired, various sequences of applying and optionally removing previously applied topical lotion and/or said second topical nanocrystal lotion may be used for various phototherapies. The testing system may be optionally included into the automated lotion spray system.

In some embodiments, the lotion emits essentially only UVA light. In other embodiments the lotion also emits visible and/or infrared radiation in combination with UV light. These latter embodiments may be advantageous where lotion is used in phototherapies for the treatment of conditions that respond to visible and infrared radiation.

The term UV light includes radiation having a peak wavelength between 160 and about 400 nm rather than visible light having a wavelength between above about 400 and below about 700 nm. UVA light has a peak wavelength between about 290 and about 400 nm. The undoped nanocrystals emit light (i.e., radiation) with a very narrow peak width due to their size rather than due to their chemical composition. Thus, in contrast to conventional bulk ceramic phosphors which emit light with a broad peak width due to their chemical composition and activator ion content, nanocrystals emit light with varying peak wavelength due to varying their size (i.e., diameter or thickness). Furthermore, some materials, such as silicon, which ordinarily do not emit light in bulk form, emit light in nanocrystal form due to the nanocrystal size. Thus, the nanocrystal size may be selected such that the they emit only UVA light, but no UVB light. Furthermore, nanocrystal size may be selected such that they emit only UVA-1, UVA-2 and/or UVA-3 light depending on the desired effect, since the peak width of the emitted UV light is narrow.

The present discussion is provided to illustrate various non-limiting designs and parameters that may be adopted for carrying out some specific exemplary types of phototherapies in accordance with the present invention.

Lupus Phototherapy:

The treatment of lupus with UV light generated by mercury vapor based light sources and long wavelength (UVA-1) passing light filters is known and is described in “Reversal of brain dysfunction with UV-A1 irradiation in a patient with systemic lupus,” Lupus. 2003;12(6):479-82; “Ultraviolet-A1 (340-400 nm) irradiation therapy in systemic lupus erythematosus,” Lupus. August 1996;5(4):269-74; “Longterm ultraviolet-A1 irradiation therapy in systemic lupus erythematosus,” J Rheumatol. June 1997;24(6):1072-4; “Ultraviolet A1 (340-400 nm) irradiation and systemic lupus erythematosus,” J Investig Dermatol Symp Proc. September 1999;4(1):79-84. Review; “Ultraviolet-A1 irradiation decreases clinical disease activity and autoantibodies in patients with systemic lupus erythematosus,” Clin Exp Rheumatol. March-April 1994;12(2):129-35, the entire disclosures of which are incorporated herein by reference. Suitable phototherapy treatment conditions, including wavelengths, intensities and exposure times are described in these references. In some preferred embodiments, the UV radiation having a wavelength of about 340 to about 400 nm is employed. Thus, the nanocrystal diameter is selected for the nanocrystals to emit light in this range by exciton recombination. Alternatively, the nanocrystal phosphor activator may be selected for the same purpose.

Psoriasis Phototherapy:

Desirable phototherapy treatment parameters for psoriasis include exposure to a narrow band emission peaking at or between 312 nm and 311 nm. Therefore the nanocrystals can be designed to deliver a narrow band of light peaking between 312 nm and 311 nm by selection of nanocrystal size for exciton recombination light emission and/or activator for phosphor light emission. This represents an improvement over psoriasis phototherapies that use mercury vapor lamps, because such lamps are primarily capable of delivering discrete spectral lines, including discrete emissions near 308 nm and 313 nm, but not in the useful ranges with peaks between 311 nm or 312 nm, which are the preferred wavelengths for psoriasis phototherapy. If desired, the lotion may contain other nanocrystals, such as metal nanocrystals or it may contain sun block ingredients which reflect or absorb wavelengths longer than the desired wavelengths. It may also contain components which reflect or absorb wavelengths shorter than the desired wavelengths, such that the lotion acts as a band pass filter. It should be noted that this function is not limited to psoriasis therapy and may be applied to other therapy areas.

In these embodiments the lotion is applied to only that part of the body suffering from the condition or in areas previously suffering or expected to suffer in the future from the condition as a preventative measure. For example, the lotion may be applied only to an arm, a leg or the face of the subject.

UV phototherapy for the treatment of psoriasis is described in U.S. Pat. Ser. No. 6,436,127, the entire disclosure of which is incorporated hereby reference. This reference includes additional descriptions of suitable phototherapy parameters, such as wavelength ranges, power and duration.

U.S. patent application Ser. No. 10/714,824 is hereby incorporated by reference in its entirety. PCT Patent Application Number PCT/US2004/014527, filed May 24, 2004 and PCT Patent Application Number PCT/US2004/016299, filed May 24, 2004 are hereby incorporated by reference in their entirety.

The foregoing description of the invention has been presented for purposes of illustration and description. It is not intended to be exhaustive or to limit the invention to the precise form disclosed, and modifications and variations are possible in light of the above teachings or may be acquired from practice of the invention. The drawings and description were chosen in order to explain the principles of the invention and its practical application. It is intended that the scope of the invention be defined by the claims appended hereto, and their equivalents.

What is claimed is:

1. A topical lotion adapted to be applied to human skin, comprising:
a carrier material adapted to be applied to human skin; and
radiation emitting nanocrystals located in the carrier material, wherein the nanocrystals are adapted to convert photons of a first wavelength range into photons of a second wavelength range suitable for phototherapy.

2. The lotion of claim 1, wherein the phototherapy is selected from a group comprising tanning, scleroderma therapy, scleriasis therapy, lupus therapy, photopheresia, or photochemotherapy.

3. The lotion of claim 2, wherein:
the phototherapy comprises tanning; and
the nanocrystals are adapted to convert radiation containing wavelengths shorter than 340 nm to radiation having a peak wavelength in a range between 340 nm and 390 nm.

4. The lotion of claim 2, wherein:
the phototherapy comprises Lupus therapy; and
the nanocrystals are adapted to convert radiation containing wavelengths shorter than 340 nm to radiation having a peak wavelength in a range between 340 nm and 400 nm.

5. The lotion of claim 2, wherein:
the phototherapy comprises psoriasis therapy; and
the nanocrystals are adapted to convert radiation containing wavelengths shorter than 340 nm to radiation having a peak wavelength in a range between 308 nm and 313 nm.

6. The lotion of claim 1, wherein the lotion is located in a container adapted to dispense the lotion onto human skin.

7. The lotion of claim 1, wherein the nanocrystals comprise semiconductor nanocrystals.

8. The lotion of claim 7, wherein the nanocrystals comprise at least one of ZnO or TiO₂ nanocrystals which are adapted to emit UV radiation.

9. The lotion of claim 1, wherein:
the nanocrystals are suspended in the carrier material; and
the carrier material is substantially transparent to UV radiation.

10. The lotion of claim 9, wherein the carrier material is selected from a group consisting of petrolatum, mineral oil, silicone oil, petroleum jelly, water, alcohol or fluorocarbon material.

11. A phototherapy method comprising:
applying to skin a lotion comprising a carrier material and nanocrystals located in the carrier material; and
exposing the lotion to photons of a first wavelength range such that the nanocrystals convert the photons of a first wavelength range into photons of a second wavelength range suitable for phototherapy and provide the photons of the second wavelength range to the skin.

12. The method of claim 11, wherein the phototherapy is selected from a group comprising tanning, scleroderma therapy, scleriasis therapy, lupus therapy, photopheresia, or photochemotherapy.

13. The method of claim 12, wherein:
the phototherapy comprises tanning; and
the nanocrystals convert radiation containing wavelengths shorter than 340 nm to radiation having a peak wavelength in a range between 340 nm and 390 nm.

14. The method of claim 12, wherein:
the phototherapy comprises lupus therapy; and
the nanocrystals convert radiation containing wavelengths shorter than 340 nm to radiation having a peak wavelength in a range between 340 nm and 390 nm.

15. The method of claim 12, wherein:
the phototherapy comprises psoriasis therapy; and
the nanocrystals convert radiation containing wavelengths shorter than 340 nm to radiation having a peak wavelength in a range between 308 nm and 313 nm.

16. The method of claim 11, wherein the lotion is dispensed from a container adapted to dispense the lotion onto human skin.

17. The method of claim 11, wherein the nanocrystals comprise semiconductor nanocrystals.

18. The method of claim 17, wherein the nanocrystals comprise at least one of ZnO or TiO₂ nanocrystals which emit UV radiation upon absorbing incident radiation.

19. The method of claim 11, wherein:
the nanocrystals are suspended in the carrier material; and
the carrier material is substantially transparent to UV radiation.

20. The method of claim 19, wherein the carrier material is selected from a group consisting of petrolatum, mineral oil, silicone oil, petroleum jelly, water, alcohol or fluorocarbon material.

21. The method of claim 11, wherein the nanocrystals convert the photons of a first wavelength range into photons of a second wavelength range by absorbing incident light in the first wavelength range and emitting light in the second wavelength range.

22. The method of claim 11, wherein the skin comprises human skin.

23. The method of claim 11, wherein the step of applying comprises spraying the lotion from a pressurized lotion container.

24. The method of claim 23, wherein the container comprises a hand-held spray container or a container which is part of a spray tanning chamber.
